(19) United States
(12) Patent Application Publication
(10) Pub. No.: US 2023/0338075 A1
(43) Pub. Date: Oct. 26, 2023

(54) IMPLANT EXTRACTOR

(71) Applicant: Shukla Medical, St. Petersburg, FL (US)

(72) Inventor: Nicholas Christopher Keach, Lutz, FL (US)

(73) Assignee: Shukla Medical, St. Petersburg, FL (US)

(21) Appl. No.: 17/726,700

(22) Filed: Apr. 22, 2022

(51) Int. Cl. A61B 17/92; A61F 2/46

(52) U.S. Cl. CPC A61B 17/92; A61F 2/461; A61B 2017/00477

(57) ABSTRACT

A implant extractor for removing an implant is provided. The implant extractor includes an extractor head having a laterally extending blade, an intermediate portion adjacent to the blade, and a laterally extending strike plate adjacent to the intermediate portion, the blade including a proximal side for mating with the implant; a shaft having proximal and distal ends, the distal end of the shaft being coupled to the extractor head; and a handle coupled to the proximal end of the shaft.

### BACKGROUND OF THE INVENTION

Various medical conditions, such as arthritis, may necessitate joint arthroplasty whereby a surgeon implants artificial structures (known as implants) within a human joint, such as a knee joint, to compensate for degradation in structure or function of the joint. Depending on the level of degradation, a full or partial joint arthroplasty may be necessary. Implants are typically press-fit onto bone within the joint or affixed to bone using a fast-acting cement, such as polymethylmethacrylate. In some instances, it may be advantageous to remove or replace an implant, for instance, when the implant wears out or the joint becomes infected.

### BRIEF SUMMARY OF THE INVENTION

In accordance with an exemplary embodiment of the subject disclosure, an implant extractor for removing an implant is provided. The implant extractor includes an extractor head having a laterally extending blade having a proximal side for mating with the implant, an intermediate portion adjacent to the blade, and a laterally extending strike plate adjacent to the intermediate portion; a shaft having a distal end coupled to the extractor head and a proximal end opposite the distal end; and a handle coupled to the proximal end of the shaft.

In accordance with one aspect of the subject disclosure, the proximal side of the blade is completely planar.

In accordance with another aspect of the subject disclosure, the blade is an elongated blade.

In accordance with still another aspect of the subject disclosure, the blade is an elongated tapered blade.

In accordance with yet another aspect of the subject disclosure, the blade comprises a distal side having a beveled surface.

In accordance with still another aspect of the subject disclosure, the beveled surface extends along lateral edges of the blade.

In accordance with yet another aspect of the subject disclosure, the blade comprises a rounded tip about its laterally extending end.

In accordance with still another aspect of the subject disclosure, the strike plate is substantially prism-shaped.

In accordance with yet another aspect of the subject disclosure, the extractor head has an overall length of about 60 to 63 mm.

In accordance with still another aspect of the subject disclosure, the blade has a longitudinal length of about 29 to 30 mm, the intermediate portion has a longitudinal length of about 22 to 23 mm, and the strike plate has longitudinal length of about 9 to 10 mm.

In accordance with yet another aspect of the subject disclosure, the intermediate portion includes a distally facing planar surface.

In accordance with still another aspect of the subject disclosure, the blade further includes a transition surface curving distally from the distal side and terminating at the distally facing planar surface of the intermediate portion.

In accordance with yet another aspect of the subject disclosure, the shaft includes a key, and wherein the handle is removably couplable to the shaft via the key.

In accordance with still another aspect of the subject disclosure, the shaft includes a key about ⅔ of an overall length of the shaft from the distal end of the shaft.

In accordance with yet another aspect of the subject disclosure, the shaft includes a substantially plus-shaped key about its proximal end for engaging the handle.

In accordance with still another aspect of the subject disclosure, the substantially plus-shaped key is an elongated key having a beveled proximal end.

In accordance with yet another aspect of the subject disclosure, the distal end of the shaft includes screw threads for coupling the shaft to the extractor head.

In accordance with still another aspect of the subject disclosure, a proximal end of the handle includes a strike plate.

In accordance with yet another aspect of the subject disclosure, a distal end of the handle is spaced from the extractor head about 276 to 294 mm.

In accordance with another exemplary embodiment of the subject disclosure, an implant extractor for removing an implant is provided. The implant extractor has an extractor head that includes a laterally extending elongated tapered blade having a planar proximal side for mating with the implant, a distal side having a beveled surface extending along lateral edges of the blade, and a rounded tip about its laterally extending end, an intermediate portion adjacent to the blade, the intermedia portion having a distally facing planar surface, wherein the blade includes a transition surface curving distally from the distal side and terminating at the distally facing planar surface of the intermediate portion, and a laterally extending prism-shaped strike plate adjacent to the intermediate portion; a shaft having a distal end coupled to the extractor head, a proximal end opposite the distal end, and a key spaced from the distal end about ⅔ of an overall length of the shaft; and a handle coupled to the proximal end of the shaft about the key and having a strike plate about its proximal end.

### BRIEF DESCRIPTION OF THE SEVERAL VIEWS OF THE DRAWINGS

The following detailed description of an exemplary embodiment of the subject disclosure will be better understood when read in conjunction with the appended drawings. For the purpose of illustrating the present disclosure, there is shown in the drawings an exemplary embodiment. It should be understood, however, that the subject application is not limited to the precise arrangements and instrumentalities shown.

FIG. 1 is a perspective view of an implant extractor in accordance with an exemplary embodiment of the subject disclosure;

FIG. 2 is an exploded perspective view of the implant extractor of FIG. 1;

FIG. 3 is a side view of an extractor head and shaft of the implant extractor of FIG. 1;

FIG. 4 is a distally facing view of the implant extractor of FIG. 1;

FIG. 5 is a perspective view of an extractor head and shaft of the implant extractor of FIG. 1;

FIG. 6 is another perspective view of the extractor head and shaft of the implant extractor of FIG. 1;

FIG. 7 is a side view of the implant extractor of FIG. 1 positioned adjacent to an implant to be removed from bone;

FIG. 8 is a side view of the implant extractor of FIG. 1 showing a blade of the implant extractor driven partially into a boundary between an implant and bone;

FIG. 9 is a side view of the implant extractor of FIG. 1 shown prying an implant off of bone; and

FIG. 10 is a flow diagram detailing a process for using the implant extractor of FIG. 1 to remove an implant from bone.

### DETAILED DESCRIPTION OF THE INVENTION

Reference will now be made in detail to an exemplary embodiment of the subject disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as upper, lower, top, bottom, above, below and diagonal, are used with respect to the accompanying drawings. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art.

“Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more exemplary embodiments. One skilled in the relevant art will recognize, in light of the description herein, that the subject disclosure can be practiced without one or more of the specific features or advantages of a particular exemplary embodiment. In other instances, additional features and advantages may be recognized in certain exemplary embodiments that may not be present in all exemplary embodiments of the present disclosure.

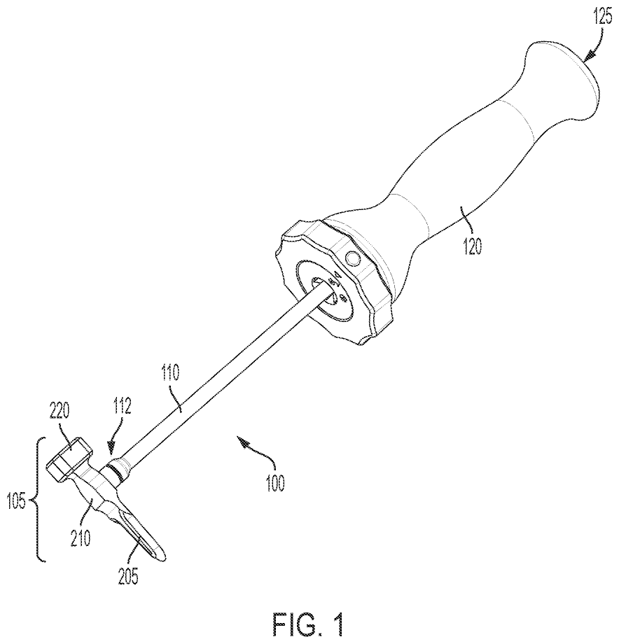

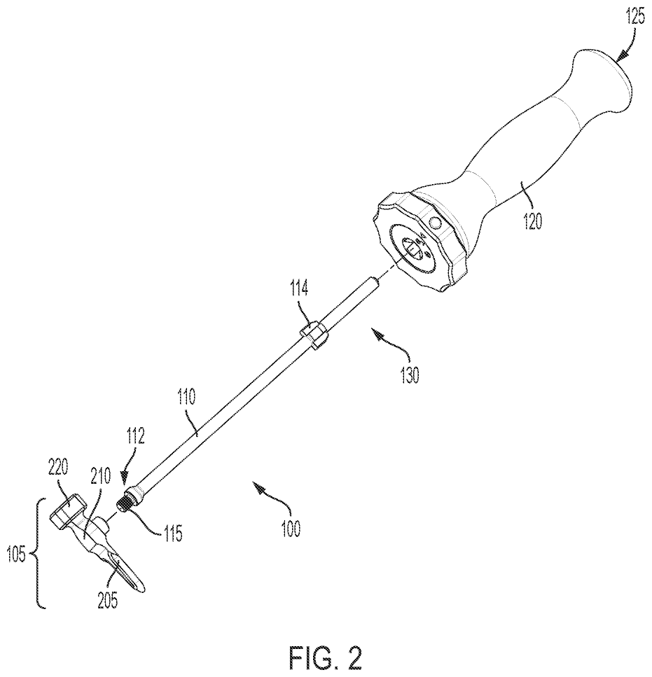

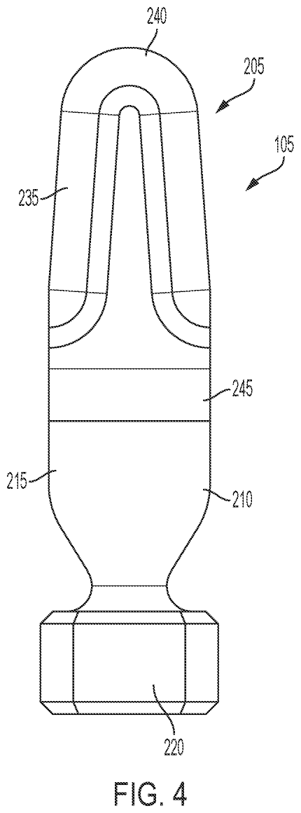

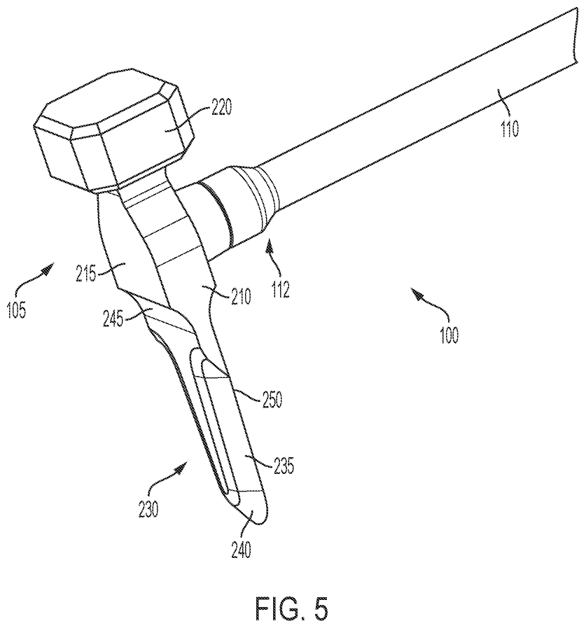

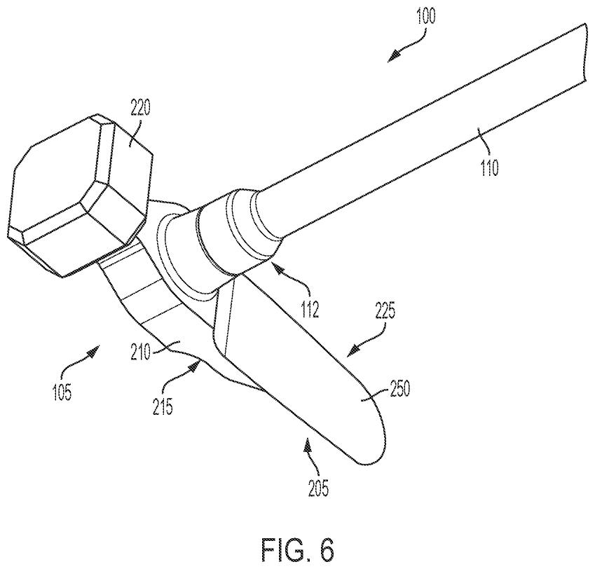

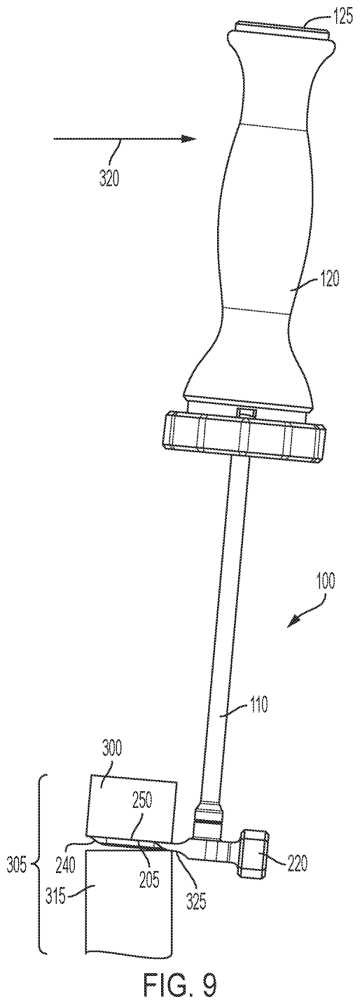

Referring now to FIGS. 1-9, there is shown an implant extractor 100 in accordance with an exemplary embodiment of the subject disclosure. Implant extractor 100 may be used, for example, to extract structures implanted within or adjacent to bone, such as, for example, a partial or uni-compartmental knee implant. It should be appreciated that implant extractor 100 may be employed also to remove other types of implants (such as a total knee, hip or shoulder implants) and/or other implants installed at other locations in a human body, such as, for example, in an elbow joint, shoulder joint, etc.

As best shown in FIGS. 1-6, implant extractor 100 includes an extractor head 105, a shaft 110 coupled at a proximal end 112 to extractor head 105, and a handle 120 with a strike plate 125 coupled to a proximal end 130 of shaft 110. In the embodiments illustrated in the Figures, shaft 110 is coupled to extractor head 105 via screw threads 115, though it should be appreciated that shaft 110 may be formed integrally with extractor head 105 or coupled to extractor head 105 in other ways, such as via screws, blots, cement, or the like. It should also be appreciated that implant extractor 100 may be constructed from any materials suitable for its intended use, such as, for example, stainless steel, titanium, cobalt chrome, various combinations of these and/or other materials, etc.

Shaft 110 of implant extractor 100 further includes key 114 structured to permit shaft 110 to be removably coupled to handle 120. In the exemplary embodiments illustrated in the Figures, shaft 110 with key 114 is structured to removably couple to one or more handles as disclosed in U.S. Patent Pub. No. 2020/0060693 A1 to Sweitzer, the entire contents of which are expressly herein incorporated by reference for all purposes. It should be appreciated, however, that shaft 110 may alternatively be formed integrally with handle 120 and/or be structured to couple to different handles (including those without strike plates) or to other structures. Further, in at least some embodiments, key 114 is positioned at a position about two-thirds of an overall length of shaft 110 from the distal end of the shaft, but can be more or less e.g., +/−⅙ from said two-thirds position. The key 114 can also be substantially plus-shaped, but can also take on additional shapes suitable for its intended purpose. The key 114 can also be an elongated key having a beveled proximal end.

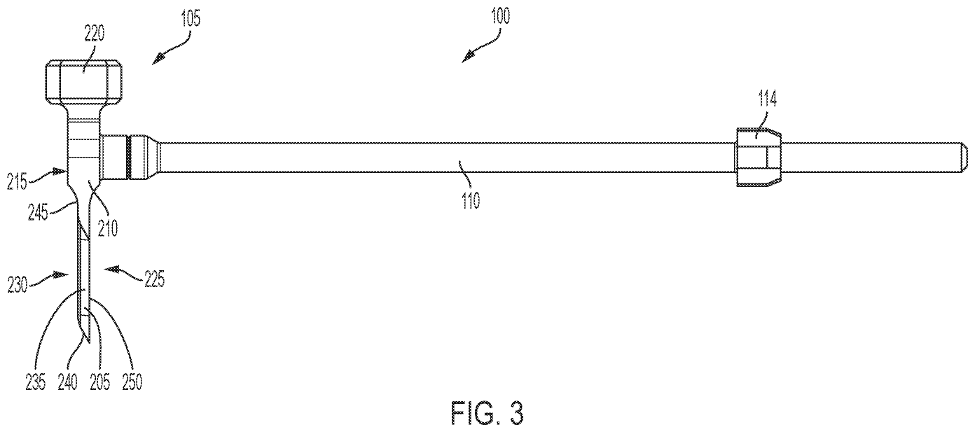

As best shown in FIGS. 3-6, extractor head 105 includes an elongated and tapered blade 205 extending laterally relative to shaft 110, an intermediate portion 210 with a distally facing planar surface 215 adjacent to blade 205 and a laterally facing strike plate 220 (which may be substantially prism-shaped) adjacent to intermediate portion 210. In the embodiments illustrated in the Figures, blade 205, intermediate portion 210 and strike plate 220 of extractor head 105 are formed integrally into one structure, though it should be appreciated that extractor head 105 may be formed from multiple separate structures coupled to one another, for example, via cement, screws, bolts, or the like. Further, in at least some embodiments, extractor head 105 has an overall longitudinal length of about 60 to 63 mm, including 61.91 mm, intermediate portion has a longitudinal length of about 22 to 23 mm including 22.54 mm, blade 205 has a longitudinal length of about 29 to 30 mm, including 29.85 mm and/or strike plate 220 has a longitudinal length of about 9 to 10 mm, including 9.53 mm, though it should be appreciated that any or all of these features may be dimensioned differently. In accordance with another aspect, a distal end or most distally extending end of handle 120 is spaced from extractor head 105 about 276 to 294 mm, including 293.2 mm, though it should be appreciated that different spacings may be provided.

Blade 205 includes a proximal side 225 for mating with an implant to be extracted and a distal side 230 having a beveled surface 235 extending along the lateral edges of blade 205, a rounded tip 240 about a laterally extending end, and a transition surface 245 curving distally and terminating at planar surface 215 of intermediate portion 210. In the illustrated embodiments, proximal side 225 of blade 205 includes a planar mating surface 250 structured to mate with a corresponding planar portion of an implant, though it should be appreciated that mating surface 250 may be structured and/or shaped differently to mate with one or more differently shaped surfaces of an implant.

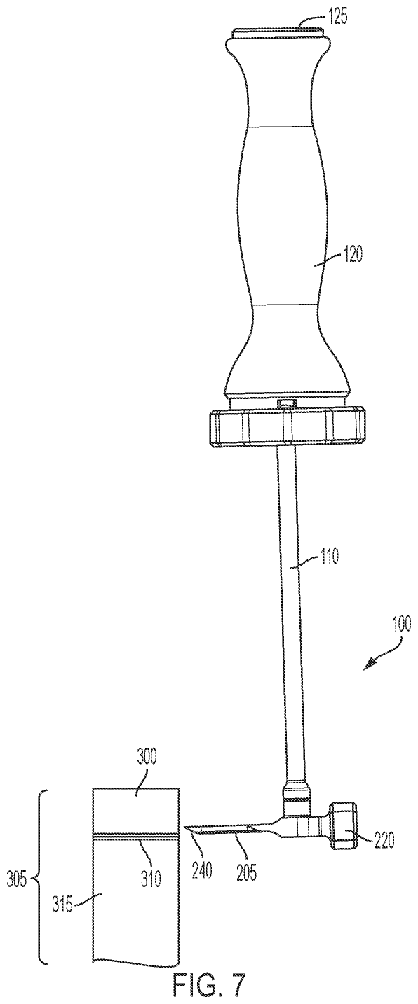

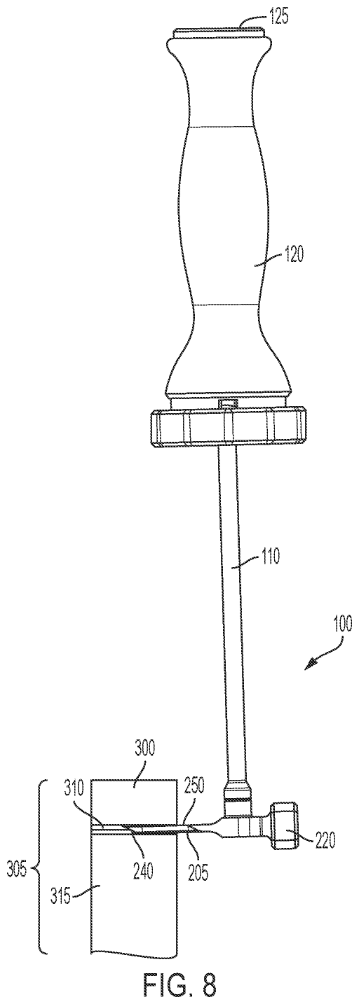

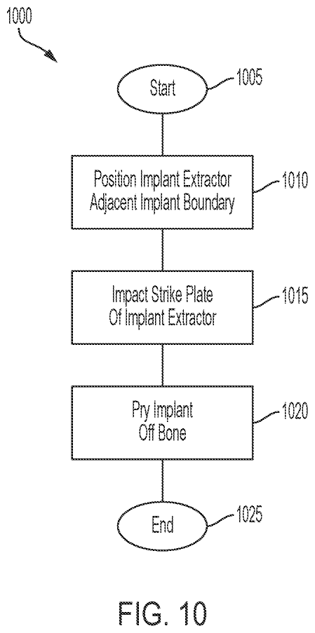

Referring now to FIG. 10, there is seen a flow diagram detailing a process 1000 for using implant extractor 100 to remove a partial knee implant 300 from e.g. a human knee joint 305. Process 1000 begins at step 1005 and proceeds to step 1010, at which a user (such as a surgeon) positions blade 205 of implant extractor 100 adjacent to a boundary 310 (with or without cement) between partial knee implant 300 and a leg bone 315 (see FIG. 7). The process then proceeds to step 1015. At this step, the surgeon impacts strike plate 220 of implant extractor 100 using a mallet, hammer or other suitable device. The impact force of the mallet is channeled through blade 205 and focused by rounded tip 240 to drive blade 205 of implant extractor 100 into boundary 310 between partial knee implant 300 and leg bone 315 (see FIG. 8). As best shown in FIG. 8, beveled surface 235 of blade 205 helps maintain planar mating surface 250 of blade 205 against implant 300 while blade 205 is driven into boundary 310.

After blade 205 is driven a sufficient distance into boundary 310 between implant 300 and bone 315, the process proceeds to step 1020. At this step, the surgeon applies a lateral force 320 to handle 120 to pivot implant extractor 100 about contact point 325 and pry implant 300 off bone 310. In addition to or in lieu of applying lateral force 320, the surgeon may use a mallet, hammer or the like to strike and/or impact strike plate 125 of handle 120 to pivot implant extractor 100. The process then ends at step 1025.

While FIG. 10 describes process 1000 for employing implant extractor 100 to remove a partial knee implant 300, it should be appreciated that implant extractor 100 may be employed to remove other types of implants, such as total knee implants or implants installed at other locations within a human body.

It should also be appreciated by those skilled in the art that changes may be made to the exemplary embodiments described above without departing from the broad inventive concept thereof. It is to be understood, therefore, that this disclosure is not limited to the particular exemplary embodiments disclosed, but it is intended to cover modifications within the spirit and scope of the claims defined herein.

I/We claim:

1. A implant extractor for removing an implant, comprising:
an extractor head having:
a laterally extending blade having a proximal side for mating with the implant,
an intermediate portion adjacent to the blade, and
a laterally extending strike plate adjacent to the intermediate portion;
a shaft having a distal end coupled to the extractor head and a proximal end opposite the distal end; and
a handle coupled to the proximal end of the shaft.

2. The implant extractor of claim 1, wherein the proximal side of the blade is completely planar.

3. The implant extractor of claim 1, wherein the blade is an elongated blade.

4. The implant extractor of claim 1, wherein the blade is an elongated tapered blade.

5. The implant extractor of claim 1, wherein the blade comprises a distal side having a beveled surface.

6. The implant extractor of claim 5, wherein the beveled surface extends along lateral edges of the blade.

7. The implant extractor of claim 1, wherein the blade comprises a rounded tip about its laterally extending end.

8. The implant extractor of claim 1, wherein the strike plate is substantially prism-shaped.

9. The implant extractor of claim 1, wherein the extractor head has an overall length of about 60 to 63 mm.

10. The implant extractor of claim 1, wherein the blade has a longitudinal length of about 29 to 30 mm, the intermediate portion has a longitudinal length of about 22 to 23 mm, and the strike plate has longitudinal length of about 9 to 10 mm.

11. The implant extractor of claim 1, wherein the intermediate portion includes a distally facing planar surface.

12. The implant extractor of claim 11, wherein the blade further includes a transition surface curving distally from the distal side and terminating at the distally facing planar surface of the intermediate portion.

13. The implant extractor of claim 1, wherein the shaft includes a key, and wherein the handle is removably couplable to the shaft via the key.

14. The implant extractor of claim 1, wherein the shaft includes a key about ⅔ of an overall length of the shaft from the distal end of the shaft.

15. The implant extractor of claim 1, wherein the shaft includes a substantially plus-shaped key about its proximal end for engaging the handle.

16. The implant extractor of claim 15, wherein the substantially plus-shaped key is an elongated key having a beveled proximal end.

17. The implant extractor of claim 1, wherein the distal end of the shaft includes screw threads for coupling the shaft to the extractor head.

18. The implant extractor of claim 1, wherein a proximal end of the handle includes a strike plate.

19. The implant extractor of claim 1, wherein a distal end of the handle is spaced from the extractor head about 276 to 294 mm.

20. A implant extractor for removing an implant, comprising:
an extractor head that includes:
a laterally extending elongated tapered blade having:
a planar proximal side for mating with the implant,
a distal side having a beveled surface extending along lateral edges of the blade, and
a rounded tip about its laterally extending end,
an intermediate portion adjacent to the blade, the intermedia portion having a distally facing planar surface, wherein the blade includes a transition surface curving distally from the distal side and terminating at the distally facing planar surface of the intermediate portion, and
a laterally extending prism-shaped strike plate adjacent to the intermediate portion;
a shaft having:
a distal end coupled to the extractor head,
a proximal end opposite the distal end, and
a key spaced from the distal end about ⅔ of an overall length of the shaft; and
a handle coupled to the proximal end of the shaft about the key and having a strike plate about its proximal end.
